(19) United States
(12) Patent Application Publication
(10) Pub. No.: US 2013/0338591 A1
(43) Pub. Date: Dec. 19, 2013

(54) PNEUMATIC PUMP OPERABLE WITHOUT ELECTRICITY, PUMPING APPARATUS, AND APPLICATIONS THEREOF

(71) Applicant: University of Louisville Research Foundation, Inc., Louisville, KY (US)

(72) Inventors: Shamus McNamara, New Salisbury, IN (US); William D. Ehringer, Charlestown, KY (US); Stephanie Miles, Louisville, KY (US); Alex Bell, Lebanon Junction, KY (US); Kunal Pharas, Boise, ID (US)

(73) Assignee: University of Louisville Research Foundation, Inc., Louisville, KY (US)

(21) Appl. No.: 13/921,734

(22) Filed: Jun. 19, 2013

Related U.S. Application Data
(60) Provisional application No. 61/661,525, filed on Jun. 19, 2012.

(51) Int. Cl. A61M 5/142

(52) U.S. Cl. CPC A61M 5/14212
USPC 604/141

(57) ABSTRACT

Infusion pumps that do not require electricity for operation are disclosed for various applications, including but not limited to pharmaceutical delivery of medications. A substance, which can be liquid, is stored in a reservoir and driven pneumatically by pressure generated by thermal transpiration, which requires a temperature difference existing between two ends of a narrow channel. In the case of pharmaceutical delivery, the medication is delivered to the treatment site via an applicator. Power sources other than electricity include, but are not limited to, the temperature of a person's body heat exceeding the ambient temperature of surrounding air.

### CROSS REFERENCE TO RELATED APPLICATIONS

This application claims priority to U.S. Provisional Application No. 61/661,525, filed on Jun. 19, 2012, the teachings and entire disclosure of which are fully incorporated herein by reference.

### GOVERNMENT RIGHTS

This invention was made with Government support under Grant No. EPS-0814194 awarded by the National Science Foundation (NSF), as well as NSF Award CBET-1133877. The Government has certain rights in the invention.

### FIELD OF INVENTION

Present embodiments relate to infusion pumps for delivery of medications and other substances, which are operable without the use of electricity.

### BACKGROUND

Infusion pumps are commonly used for a variety of applications, including but not limited to medical, home care, and ambulatory. One of many uses is for drug delivery. There are various fluid control mechanisms for infusion pumps used in drug delivery. Examples include, but are not limited to, elastomeric pumps which store the drug in a reservoir, generally in liquid form, and the drug is driven from the reservoir via tubing by external pressure applied to the reservoir.

Second, syringe-based pumps store drug in a syringe. The syringe has a reservoir (sometimes referred to as a barrel) for storing the drug, and a plunger. As the syringe plunger is pushed at a particular rate, it drives the fluid from the reservoir through tubing attached to the reservoir.

A third example is gravity-based, in which a reservoir—which can be a flexible bag holding the drug—is suspended in the air, and the infusion pump is configured to resist the flow of the drug from the reservoir into attached tubing. In all three examples, one end of the tubing serves as an outlet from the reservoir, allowing the drug to exit from the reservoir. The opposite end is a tubing outlet for delivery of drug to the patient, e.g., intravenously or in direct contact with the skin.

Regardless of which fluid control mechanism is selected, conventional infusion pumps require a source of electricity, which is usually provided by either AC power or batteries. Consequently, the use of infusion pumps could become impractical if electricity is unavailable or scarce. Combat situations, catastrophic weather occurrences, and other emergency situations are examples where conventional infusion pumps could become useless. In such cases, the ability to successfully treat the patient could depend on having an alternative to conventional infusion pumps that require electricity.

A Knudsen pump is a type of infusion pump. Such a pump operates based on thermal transpiration, in which a pressure gradient is created when a temperature difference exists at opposite ends of a narrow channel. One end of this channel is referred to as the “hot side”, and the other end is referred to as the “cold side.” As the temperature at the hot side increases, the density of gases (e.g., air) decreases there. Conversely, the lower the temperature at the cold side, the greater the density there. The increased density at the cold side causes more air to flow into the channel toward the hot side. The result is a pneumatic flow of air from the cold side to the hot side, thus supplying pressure in the direction of flow.

Beneficially, most Knudsen pumps feature no moving parts that may fail. However, the use of such pumps to consistently and reliably operate, without electricity, depends on generating sufficient heat to establish the necessary temperature difference between the hot side and the cold side. Thus, establishing and maintaining adequate temperature differences between the hot side and the cold side is a significant challenge in the technology of Knudsen pumps. Although such pumps are known, there are many reasons why they might not function satisfactorily, for example if the temperature difference between the hot side and the cold side is only slight. As discussed further below, determining the temperature difference necessary for adequate pumping depends on factors such as the dimensions of the channel, or channels, and other configurable features of the pump.

In short, a substantial need exists for infusion pumps that operate pneumatically without electricity, based on the principle of thermal transpiration, and which are capable of various uses including but not limited to drug delivery. Advantageously, as described and claimed herein, multiple embodiments of such a pump are capable of producing a steady, regular, relatively low flow of liquid on the order of about 5 cm³/min per cm² of membrane area, which is due to thermal transpiration based on the difference between normal body temperature (hot side of channel) compared to ambient temperature of about 20° C.

Specifically, the temperature difference is a person's body temperature at the skin compared to the ambient temperature of the surrounding atmosphere. As described and claimed herein, such embodiments are easy to use, and they provide increased mobility for the user. Besides body heat, solar energy and heat sources can be used for creating the temperature difference between a hot side of the pump and the cold side.

There are numerous applications of the embodiments and alternative embodiments described and claimed here. Some of these include drug delivery through use of a pump that does not require electricity, and thus does not require a power cord or batteries that must be changed recurrently. In some embodiments, the drug or other substance is driven pneumatically from a reservoir through an applicator. Such a pump is configurable in multiple ways to supply varying degrees of pressure and flow rate depending on the nature of the reservoir and the type of medication.

Optionally, the pump is part of a device worn by a person, for example strapped to an arm, and power is provided by the person's body heat, or, alternatively, solar or other source of thermal energy, e.g., fire. Although many drug delivery uses are contemplated, one such use is for the treatment of dermal wounds, including but not limited to burns, pressure ulcers (commonly referred to as bed sores), and diabetic ulcers, or other conditions in which a continuous supply of medication facilitates treatment and healing. Methods of drug administration using the embodiments herein include, but are not limited to, intravenous, intramuscular, topical, transdermal, and subcutaneous. Besides drug delivery, the embodiments described and claimed herein are useful in other applications, as well, e.g., for gas chromatography and lab-on-a-chip applications.

### BRIEF DESCRIPTION OF THE DRAWINGS

The written description of multiple embodiments and alternatives, as set forth herein, will be better understood when read in connection with the appended drawings. It will be understood that the embodiments are not limited to precise arrangements shown, nor are the illustrations intended as being drawn to scale.

FIG. 1 is a schematic view of the principle of operation of a pneumatic pump operable without electricity, according to multiple embodiments and alternatives.

FIG. 2A is a top perspective view of a pneumatic pump operable without electricity and pumping apparatus, with cutaway showing interior positioning of reservoir, according to multiple embodiments and alternatives.

FIG. 2B-2D show scanning electron micrographs depicting pores of three different membranes for a pneumatic pump operable without electricity, according to multiple embodiments and alternatives.

FIG. 3 is an exploded top perspective view of a pneumatic pump operable without electricity, and other components of a pumping apparatus, according to multiple embodiments and alternatives.

FIG. 4 is a perspective view of a pneumatic pump operable without electricity and pumping apparatus shown in cross-section, according to multiple embodiments and alternatives.

FIG. 5 is an assembled top perspective view of a pneumatic pump operable without electricity and other components of a pumping apparatus, with cutaway view of a portion of the interior, according to multiple embodiments and alternatives.

FIG. 6 is a top perspective of a device comprising a pneumatic pump operable without electricity and other components integrated with such a device, according to multiple embodiments and alternatives.

FIG. 7 is a graph showing pressure charted against time for two pumps combined in series, compared to the two pumps individually, according to multiple embodiments and alternatives.

FIG. 8 shows a thermal circuit for analyzing AT calculations, according to multiple embodiments and alternatives described herein.

FIG. 9 is a graph showing experimental pressure and temperature data compared to simulation based on thermal circuit analysis of pumps fabricated according to multiple embodiments and alternatives.

### MULTIPLE EMBODIMENTS AND ALTERNATIVES

A pneumatic micro gas pump operable without electricity, according to multiple embodiments and alternatives, is described and claimed. Hereafter, such a pneumatic pump is referred to as “pump,” and as “pumps” when referring to more than one. The pump comprises at least one channel with a first end and a second end, the second end being positioned closer to a heat source than the first end; the at least one channel being configured to accept a gas from a pump inlet and allow it to enter the first end and travel through the length of the channel and exit the channel at the second end. The first end corresponds to a cold side of the pump, and the second end corresponds to a hot side of the pump, the hot and cold sides respectively being determined according to a temperature difference across the channel.

A pumping apparatus includes such a pump powered by a source other than electricity, comprising at least one narrow channel through which matter in the form of gas passes, a reservoir in fluid communication with the pump, which is for storing a substance, and an applicator. In some embodiments, multiple channels are provided within a membrane, e.g., a nanoporous membrane with average channel diameter no greater than about 100 nm. In some embodiments, the reservoir provides an enclosed or partially enclosed space for releasably storing a drug, and the pump pushes gas into the reservoir to drive the drug out of the reservoir into the applicator for drug delivery.

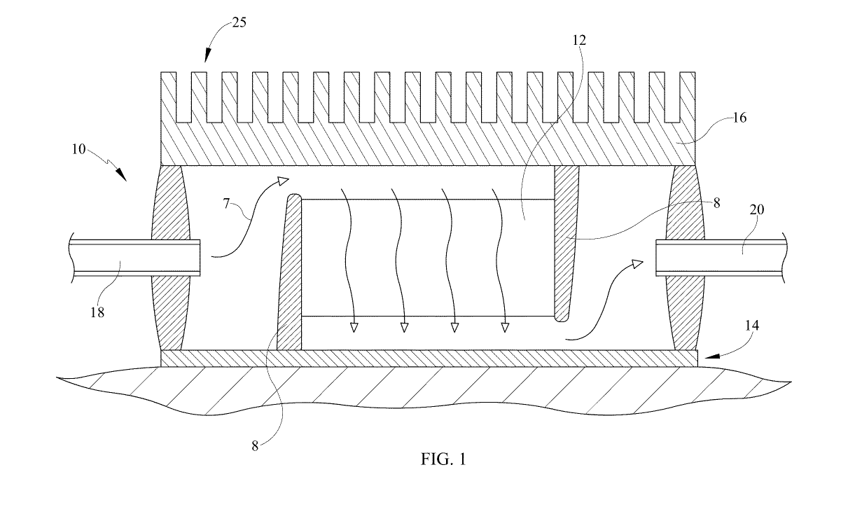

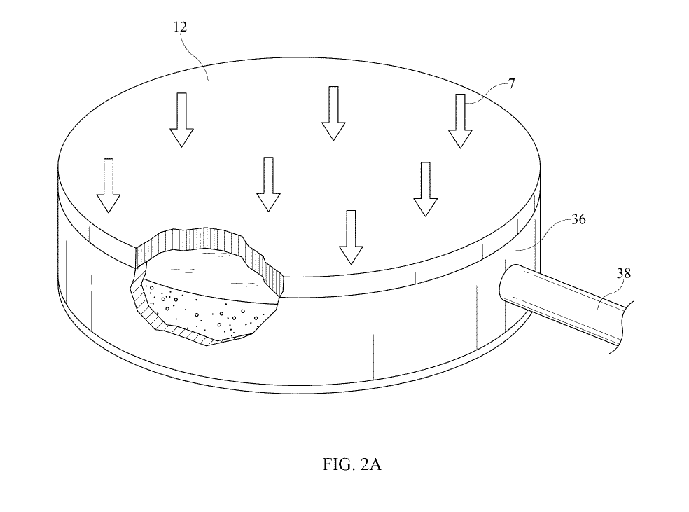

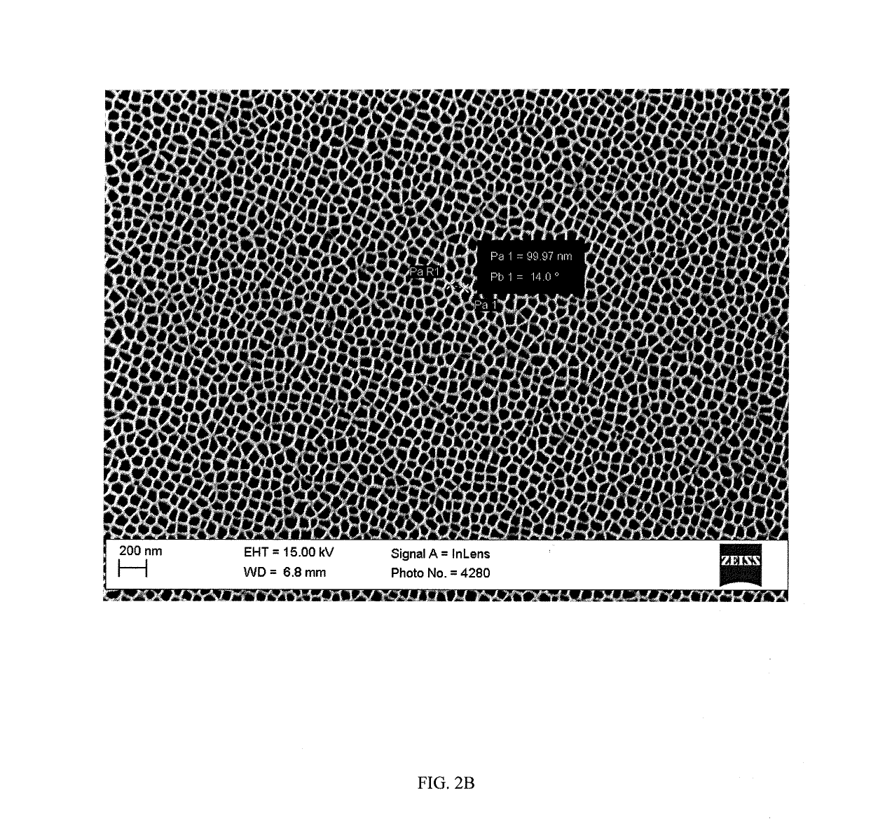

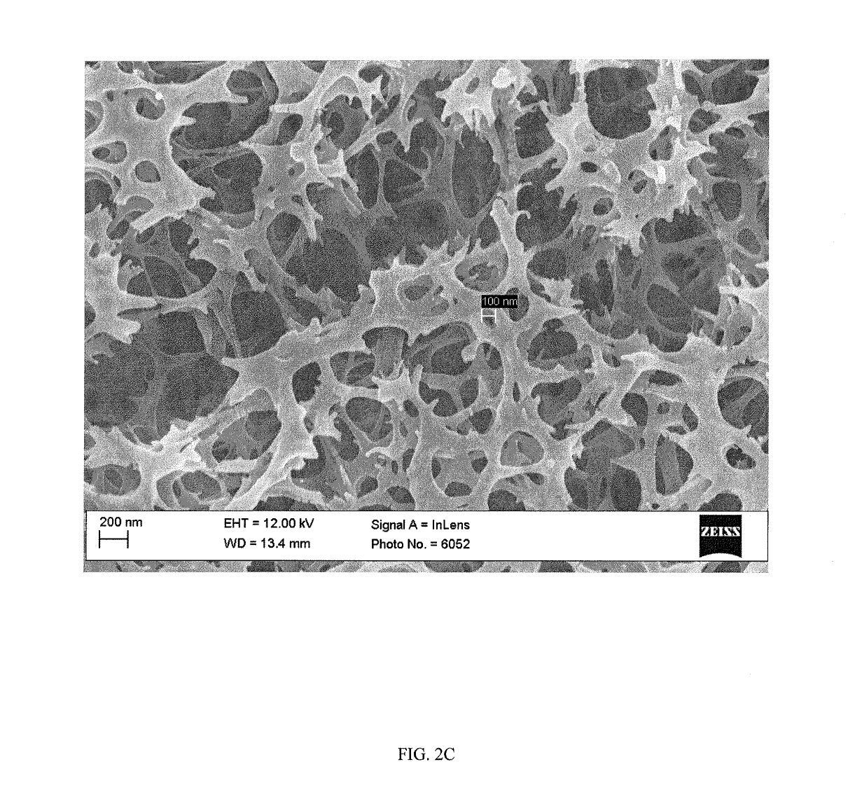

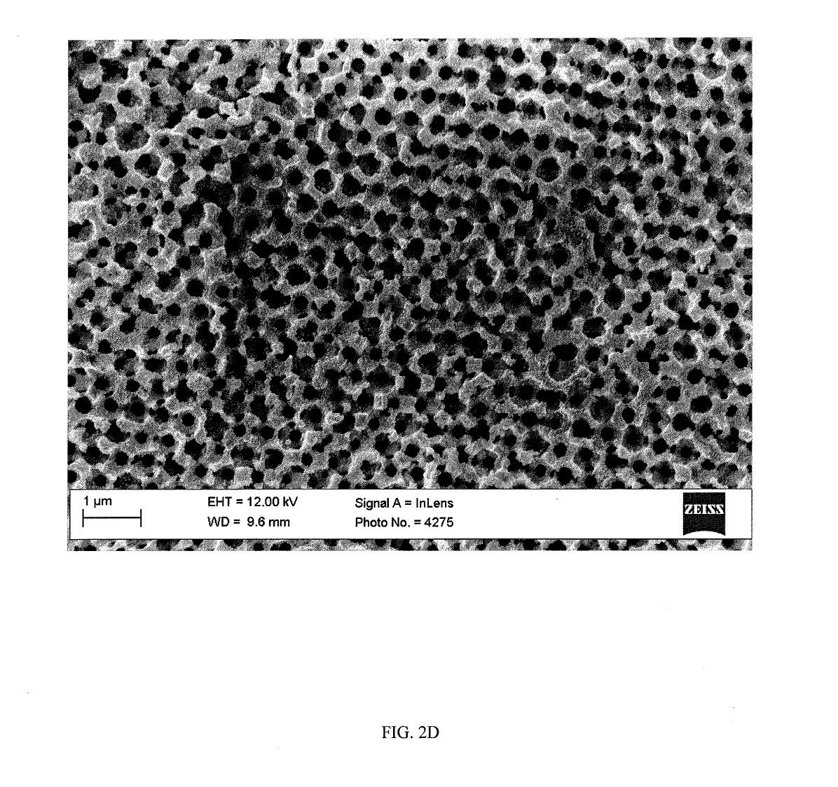

The pump operates principally by thermal transpiration, in which a pressure gradient is created when a temperature difference exists at opposite ends of a sufficiently narrow channel. According to an embodiment shown in FIG. 1, the temperature difference exists or is caused to exist across a membrane 12 within a housing 10, and having at least one channel (see FIGS. 2B-2D) through the entire thickness of the material. As the temperature at the hot side 14 of the channel increases, the density of gases (e.g., air) decreases there. Conversely, the lower the temperature at the cold side 16, the greater the density there. The increased density at the cold side 16 causes more air to enter the housing 10 via inlet 18 and then flow into the at least one channel toward the hot side 14. This results in pneumatic flow of gas from the cold side 16 to the hot side 14 and ultimately exiting housing 10 through a pump outlet 20, thus creating pneumatic pressure in the direction of flow as noted by directional arrows 7. In some embodiments, one or more barriers 8 are positioned within housing 10 for influencing direction of gas flow, e.g., from inlet 18 through channels in the direction of hot side 14 and then to outlet 20.

In some embodiments, the reservoir comprises a bag, such as that commonly referred to as an IV bag, although as used here the connotation does not limit the application to intravenous drug delivery. The pressure generated by gas flow through the pump applies pressure externally to the bag, causing drug or other substance to flow from the reservoir to the applicator. The applicator regulates and evenly distributes the flow of the substance, which in some embodiments is a drug. The substance is generally a liquid or another substance having a higher viscosity, e.g., ointment. Optionally, the bag is integrated with a rigid box serving as the reservoir, which protects the bag against unintended forces that could cause the contents to be released prematurely.

More generally, with respect to operation, the pump is intended to work at or near atmospheric pressure. The average diameter of the at least one channel is about 100 nm or less. In some embodiments, the at least one channel comprises a nanoporous membrane, having a pore size of about 25 nm.

Various factors determine the pressure gradient needed to effectively drive gas from cold side to hot side. These include the size of the channel, such as the diameter for channels having a circular cross-section, or pore size in the case of a nanoporous membrane. In general, the smaller the channel, the greater the movement of gas from cold side to hot side and thus the greater the pressure differential, provided the diameter or pore size is equal to or greater than the diameter of each gas molecule.

Membrane thickness also affects the temperature difference that is generated across the channel (hot side temperature compared to cold side temperature). In general, the thicker the membrane, the greater the temperature difference and, therefore, the greater the pressure gradient for driving gas. In some embodiments, a pumping apparatus includes a bottom metal surface in direct contact with the heat source (e.g., skin of a patient), or otherwise in closer proximity to the heat source than the pump itself. This also influences the temperature difference and thereby the pressure gradient possible through the use of the pumps. Optionally, the metal surface is formed from metal, e.g. stainless steel, which is preferably a biocompatible metal having a thermal conductivity of at least about 10 W/(m*K). As discussed below, another feature for temperature control is a heat sink 25 to dissipate heat away from the cold side, which is labeled in various figures, including FIGS. 1 and 3-6.

The operation of the pump operation can also be understood with reference to the concepts of free molecular flow, viscous flow, and transitional flow. The Knudsen number (Kn), which is the mean free path of the gas divided by the hydraulic diameter of the channel, differentiates these regimes of flow. Free molecular flow occurs for Kn>10. Viscous flow occurs for Kn<0.01. In between these regimes is the transitional flow regime. Preferably, the channels of the pump are in the free molecular flow regime. Even though free molecular flow generally produces improved pump performance compared to transitional flow, adequate flow can be achieved in the transitional flow regime, the advantage of which is this permits larger pore diameters that are easier to fabricate. However, as the Knudsen number becomes smaller, the negative impact on pump performance becomes greater.

FIG. 2 shows an embodiment in which a nanoporous membrane 12 is positioned atop a reservoir 36 used for holding the medication, with membrane 12 and reservoir 36 being operationally attached in some embodiments. Pressure from gas passing from the cold side of the membrane and exiting at the hot side of the membrane drives fluid out of the reservoir at a steady flow rate, passing through applicator 38. This type of embodiment is referred to herein as an integrated pump because the pump is positioned atop and thereby integrated with the reservoir.

In some embodiments, a pump membrane is formed from suitable materials which include, but are not necessarily limited to, polypropylene, silica, nitrocellulose, aerogel, and mixed cellulose esters such as but not limited to cellulose acetate and cellulose nitrate. Preferably, a hydrophobic material, such as polypropylene, is used for drug delivery applications with the integrated device. This is because a hydrophobic membrane or channel tends to prevent or reduce the drug from leaking out of the reservoir until a sufficient temperature difference is applied and a resultant pressure gradient is achieved sufficient to drive the drug from the reservoir.

The channels within the membrane need not have a uniform diameter, and they need not be completely straight. Some embodiments employ membranes having channels that vary in size and are randomly positioned, while for other embodiments the channels are in a fairly well defined pattern. FIGS. 2B-2D show scanning electron micrograph taken with a scanning electron microscope (SEM) of three different membranes. FIG. 2B is a SEM micrograph of a membrane formed from nanoporous silicon, having a substantially straight linear channel geometry. FIG. 2C is a SEM micrograph of a membrane formed from mixed cellulose esters. The membrane is a product known as Millipore MF series filter membrane, product #VSWP02500, having a 25 nm pore size and being formed from a mixture of cellulose acetate and cellulose nitrate. FIG. 2D is a SEM micrograph of a membrane formed from porous bismuth, which is formed by pressing molten Bismuth into a silica colloidal crystal, and dissolving the silica spheres.

In general, the pressure gradient created by the temperature difference between the hot side and the cold side of a single pump is expressed according to equation 1:

$$\frac{{P}_{h}}{{P}_{c}} = \sqrt{\frac{{T}_{h}}{{T}_{c}}} \qquad ( 1 )$$

where Pₕ is the pressure (Pa) on the hot side of the pump; P_{c} is the pressure (Pa) on the cold side; Tₕ is the temperature (K) on the hot side; and T_{c} is the temperature on the cold side. For multiple pumps in series, equation 1 is expressed as equation 1A:

$$\frac{Ph}{Pc} = {( \frac{Th}{Tc} )}^{n / 2} \qquad ( 1 A )$$

n is the number of pumps connected in series. In some embodiments, the temperature difference is established by positioning the pump in contact with or in proximity to a person's skin. Here, the pump in proximity to skin refers to placing some part of the pumping apparatus in contact with the skin. In either case (direct contact between pump and skin, or pump is in proximity to skin), the temperature difference exists between the person's skin temperature, which corresponds to the hot side of the pump, and the temperature of ambient air corresponding to the cold side. Generally, for purposes of embodiments disclosed and claimed herein, ambient air temperature is considered to range from about −20° C. to about 37° C., but this range may vary depending on environmental conditions.

The pressure generated by a pump according to these embodiments can be calculated with Equation 2:

$$P = ( \frac{( {T}_{body} - {T}_{air} ) {P}_{o}}{{T}_{air}} ) {( ( \frac{{t}_{b}}{{κ}_{b}} + \frac{{t}_{w}}{{κ}_{w}} ) {κ}_{n} + {t}_{n} )}^{- 1} {t}_{n} \qquad ( 2 )$$

T_{body} is the temperature of the body (which can be human or animal); Tₐᵢᵣ is the ambient temperature; P_{c} is the ambient pressure; t_{b} is the thickness of the bottom plate; t_{w} is the thickness of the reservoir; tₙ is the thickness of the membrane; k_{b} is the thermal conductivity of the bottom plate, k_{w} is the thermal conductivity of the reservoir, and kₙ is the thermal conductivity of the nanoporous material.

Comparatively, the pump must overcome the pressure of the channel, which is calculated according to Equation 3:

$${P}_{ch} = \frac{2 γ}{a} cos ( θ ) \qquad ( 3 )$$

a is the channel radius; γ is the surface tension of the liquid; and θ is the contact angle between the liquid and gas. If pump pressure is less than channel pressure (P<P_{ch}), then liquid will not readily flow through the channel. Once pump pressure exceeds the channel pressure (P>P_{ch}), however, the pump causes liquid to flow through the channel and progresses to a relatively constant flow as a steady state temperature difference is reached.

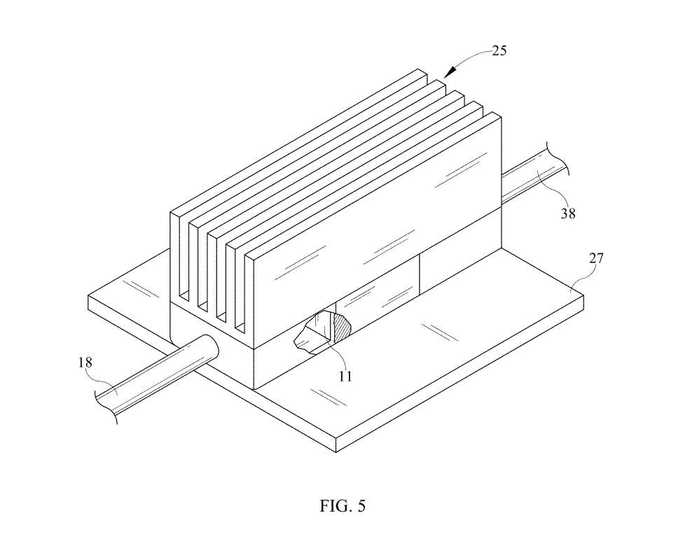

Generally, another way to increase flow rate is a configuration referred to herein as a cascaded pump. Instead of being positioned atop the reservoir, in the cascaded configuration a pump (or, pumps) is placed in closer proximity to a heat source, which in some embodiments is a person's skin. Optionally, as shown in FIG. 5, the pumping apparatus includes a bottom metal surface plate 27 positioned between the pump 11 and the heat source (not shown), and preferably directly contacting this source. Such closer proximity to the heat source, e.g., skin, increases the temperature difference attainable through use of the pump, in part because there is less thermal resistance from skin to the ambient air with the pump positioned closer to the heat source (e.g., skin). Likewise, the more channels, the greater the temperature difference and the flow rate of the pump. FIG. 5 also illustrates pump inlet 18 for accepting a gas from the atmosphere external to the pump, and outlet tubing 38 serving as an applicator for drug delivery in some embodiments.

In turn, a greater temperature difference increases the pressure gradient that can be established to drive larger volumes of gas through the channels at a faster rate. This configuration further facilities the placement of multiple pumps in series to create more pressure for driving fluid or drug from the reservoir. In general, the number of pumps placed in series increases the pressure created.

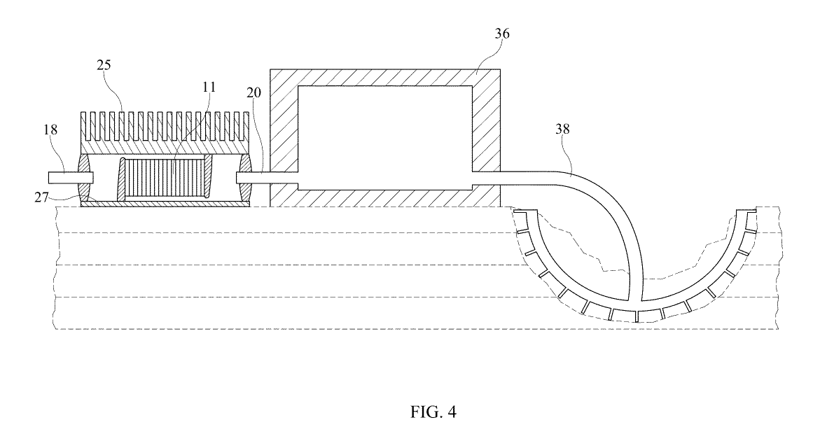

All other factors being the same, for embodiments where the pump is powered by body heat, a cascaded pump produces increased flow rate compared to an integrated pump. Largely, this is due to the cascaded pump being closer to the skin, as shown in FIG. 4. Testing of an example integrated pump produced a flow rate for the movement of air at about 156 μL/minute, where the cross-sectional area of the membrane was about 254 mm². Comparatively, a single stage cascaded pump produced a flow rate greater than 100 μL/minute, where the cross-sectional area of the membrane was about 195 mm². Another difference between the designs is for the integrated design, ΔT (temperature difference) tends to decrease as liquid is driven from the reservoir. Conversely, for the cascaded design, having the pump separate from the reservoir means the pressure is not affected by the reservoir liquid level.

Persons skilled in the relevant art will appreciate that the embodiments disclosed and claimed herein provide myriad combinations for designing a pump(s), pumping apparatus, and devices for delivering a proper dose of drug at a desirable flow rate while the pump is in use. Either mechanical or automated regulation of the flow rate may be appropriate for some uses.

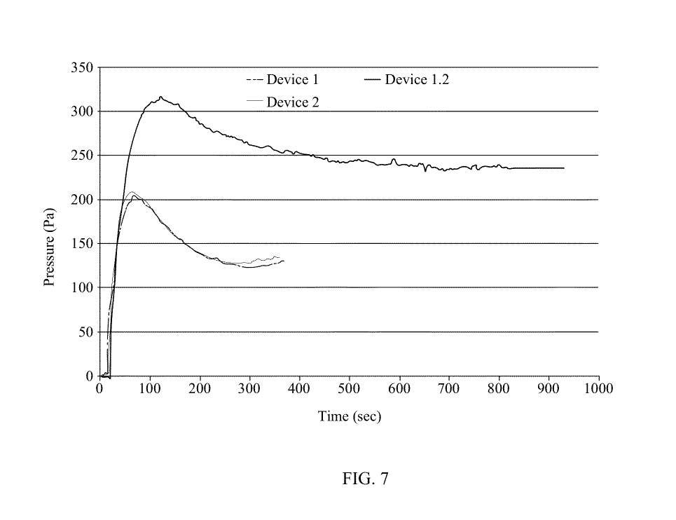

Moreover, for cascaded pump embodiments, placing multiple pumps in series will increase the pressure generated as a whole. Pumps are placed in series by attaching the outlet of a first pump to the inlet of a second pump, and so on depending on the number of pumps that are to be connected. The effect of placing pumps in series is to compound the pressure generated. FIG. 7 graphs the results of a two-stage pump consisting of pumps 1 and 2 in series, compared to pumps 1 and 2 individually. Pumps 1 and 2 were formed according to substantially the same parameters. The pump membrane for each had a cross-sectional area of 195 mm² (about 13 mm×15 mm), with a thickness of about 1.05 mm. The membrane was formed from a mixed cellulose ester with a 25 nm nominal pore diameter with 70% porosity. The overall size of the pumping apparatus was about 2070 mm³ (about 15 mm×23 mm×6 mm (w×l×h)). To assess the differences between individual performance of the pumps versus having them connected in series, gas (specifically, room air) was supplied to the pump from a syringe. A first pressure sensor was left open to ambient air, and a second pressure sensor was attached to the pump and placed at the pump outlet. These results show a greater than 50% increase of pressure for the two-stage pump.

Optionally, multiple pumps can also be connected in parallel. This is done by joining all the pumps' outlets to increase the aggregate cross-sectional area of the pump as a whole. This increases the number of channels or pores through which gas is drawn, thus increasing the flow rate. The increase will be proportional to the temperature difference between the hot side and the cold side.

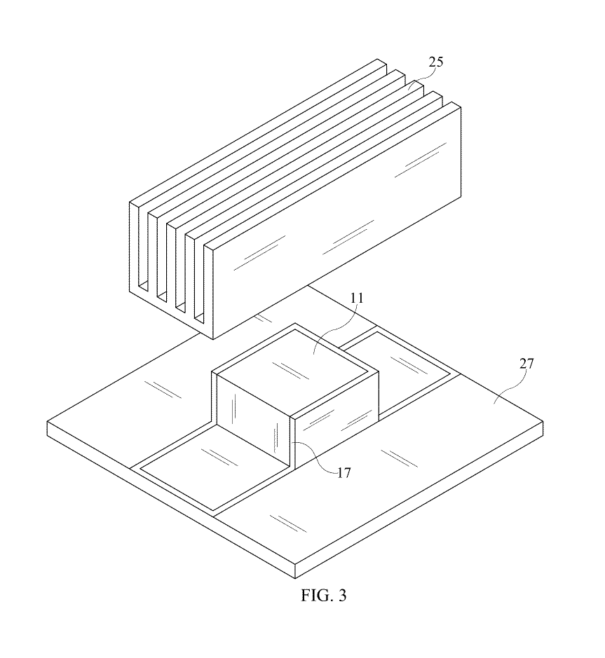

In some embodiments, a heat sink 25 is provided as shown in FIG. 3 and FIG. 5 for dissipating heat away from the cold side of the pump. Heat dissipation increases the temperature difference between the hot and cold sides, thus establishing a more robust pressure gradient for driving air toward the hot side and maintaining that gradient for a longer period of time. In some embodiments, the heat sink is formed from aluminum or other suitable metals known in the art, and preferably it is integrated into the pump itself. This is achieved by attaching the heat sink to the pump through means known in the art, for example gluing the pieces with adhesive. FIG. 3 shows an exemplary glue 17 layout in which glue is applied to the top and bottom of pump 11, which can be a nanoporous membrane, on only three sides, thus leaving one side open for gas to travel.

In another exemplary application for a pneumatic pump operable without electricity, a device that includes at least one pump and pumping apparatus is configured to be strapped to the body of a wearer. It is documented that a continuous supply of drugs may improve the healing process for a number of conditions, including but not limited to dermal wounds. The pumps and pumping apparatus described and claimed herein are useful for providing a low, consistent flow rate suited for these kinds of drug delivery applications.

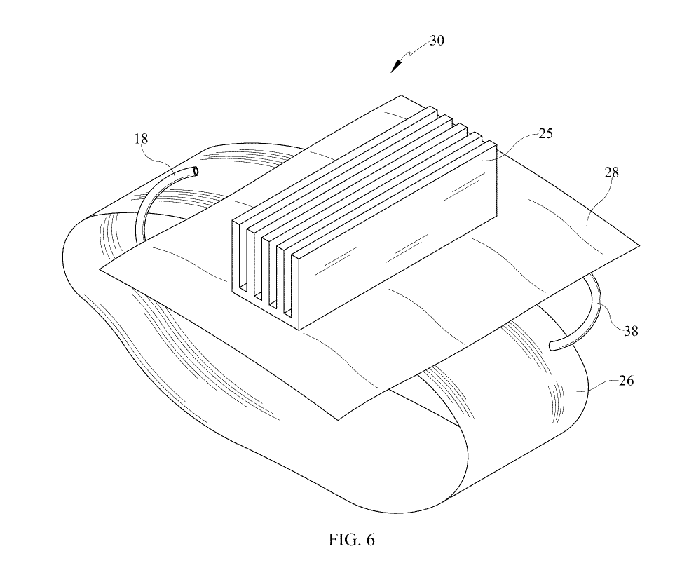

FIG. 6 shows such a wearable device 30, comprising a pneumatic pump (hidden beneath heat sink 25) and pumping apparatus operable without electricity, according to multiple embodiments herein. In an embodiment, a pump is formed from ten nanoporous membranes, each having a thickness of about 105 micrometers (μm) for a total thickness of about one millimeter (1 mm). Preferably, the membranes are sealed around the edges with adhesive, e.g., E-20 HP Hysol epoxy. The pumping apparatus further comprises a rectangular heat sink 25 formed from aluminum. In an embodiment, the heat sink is about 1.25 in.×0.53 in. (31.75 mm×13.46 mm) and about 0.19 in. thick (4.83 mm). The heat sink is partially open to ambient air and has a thermal resistance of about 34° C./W. In this embodiment, a stainless steel bottom plate 27 (best seen in FIG. 3 and FIG. 4) that is formed from 304 stainless steel with a thickness of about 0.001 in. (0.025 mm), and is configured to be positioned at or near the skin. Some embodiments include inlet tubing 18 to the pump and outlet tubing 38 as part of the pumping apparatus, the latter of which can serve as the applicator in medication delivery applications. Optionally, holes (not shown) are formed in plate 27 allowing it to be sewn into a cloth 28, which can be partially folded over to conceal the tubing of the device. Although limited to a single-stage pump in this figure, alternative embodiments will easily permit multiple pumps to be connected in series to increase the pressure gradient achievable by the pumping apparatus. As seen in the figure, one or more elastic straps 26 is optionally used for retaining the device in contact with the skin of the patient.

As will be appreciated with further reference to FIG. 5, in some embodiments both the bottom plate 27 and the aluminum heat sink 25 are initially at ambient temperature of the surrounding environment. Eventually, the temperature of the plate 27 is raised by contact or proximity with the skin of the wearer. Likewise, the temperature of the heat sink 25 is raised as thermal energy dissipates away from the cold side, eventually reaching a substantially steady state temperature difference between the hot side and cold side. Generally, as the pneumatically-driven pressure reaches and maintains a steady state, it facilitates a substantially constant flow rate.

With further reference to FIG. 4, the path of gas molecules flows from the exterior of the pumping apparatus into inlet 18. Gas molecules pass through pump 11 in the direction of the hot side of the pump, in this case being the side closer to plate 27 which is shown in contact with a user's skin (marked by dashed lines). Pump outlet 20 is in fluid communication with reservoir 36, the contents of which are driven pneumatically by the movement of the gas into and through applicator 38 where they are delivered to the treatment site, in this case depicted with dashed lines as a dermal wound.

In some embodiments, reservoir 36 comprises a box formed from suitable materials, e.g. ABS plastic, and containing two plastic IV bags. One bag holds the drug, and the second is in fluid communication with, and otherwise configured to accept a volume of gas from, the pump. As the second bag fills with gas from the pump, it applies pressure to the first bag, thereby driving the drug out of this bag. As the drug leaves the reservoir, it is delivered to the treatment site by a fluid delivery mechanism, which in this case is applicator 38 in the form of outlet tubing in fluid communication with the reservoir 36. The reservoir box is strong enough to withstand increasing pressure generated by the pump. Further, it is sufficiently rigid to protect IV bags from being pressed accidentally and releasing the drug too soon. Advantageously, a wearable infusion pump according to these embodiments is very portable and can be used for clinical, ambulatory, and home health in addition to other uses.

In some embodiments, the characteristics of the pump are adjusted to further influence flow rate. For example, increasing the thickness of the pump or membrane will increase the temperature difference, and consequently the pressure gradient will also increase. Similarly, reducing the size of the channel, or channels, increases the pressure gradient. As also discussed herein, proximity of the pump to the heat source (e.g., skin of the wearer) increases the temperature difference, in addition to these other factors. Increasing the pressure gradient in turn makes it possible to use a larger reservoir for holding more drug or other liquid that is to be moved. The embodiments described and claimed herein are not limited to a particular fluid control mechanism. Rather, various fluid control mechanisms are contemplated, including but not limited to elastomeric, syringe-based, and gravity-based.

Thermal circuit analysis is a method for predicting heat flow through a material or composite material, which is relevant to embodiments disclosed for at least two objectives. First, it is used to predict the temperature difference (ΔT) across a material, i.e., between the hot side and the cold side of a pump. Such analysis is useful for the design of the pump and for the selection of materials. Second, it is used for predicting temperature differences and effect on the pressure gradient created by changes in fluid level in the reservoir. Generally, thermal circuit analysis involves mapping thermal components to a set of mathematically analogous electrical components. For example, voltage (V) is analogous to temperature difference (ΔT) across a material; electrical resistance (R) is analogous to thermal resistance (Rₜₕ) of the material; current (I) is analogous to heat flow (Q) of the material; and electrical capacitance (C) is analogous to thermal capacitance (Cₜₕ).

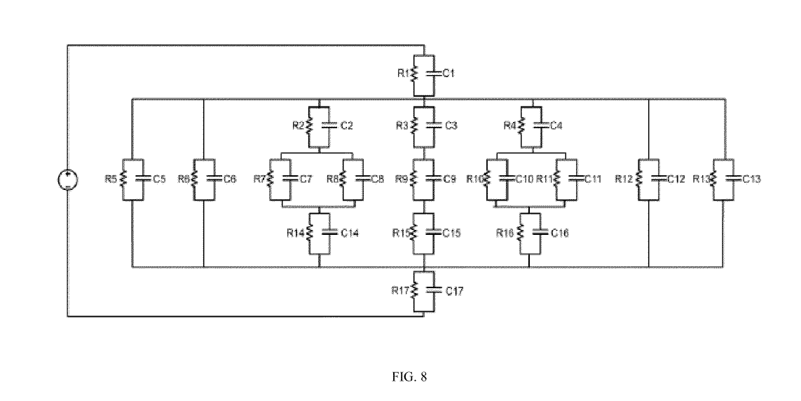

FIG. 8 shows a thermal circuit created using these analogous relationships for calculating a theoretical ΔT equation. The temperature difference across any element within the pumping apparatus is determined according to equation 4:

ΔT=Q*R (4)

Altering the materials or geometries of a pump will change the thermal resistance of the entire pumping apparatus, as well as the ΔT across the pump. Related to the ΔT equation, several other properties of a material or component of a pumping apparatus can be calculated. For a given material, thermal resistance (Rₜₕ) is calculated using equation 5:

$${R}_{th} = \frac{t}{A * K} \qquad ( 5 )$$

According to this equation, for a particular pump material or other pumping apparatus material, t is thickness; A is cross-sectional area of the pump; and K is thermal conductivity of the material. Thermal capacitance (Cₜₕ) is calculated according to equation 6:

Cₜₕ=m*cₚ (6)

where m is mass and cₚ is the specific heat of the material. The mass flow rate (M), which is the quantity of mass passing through a cross section of the channel per unit of time, is calculated using equation 7:

$$M = {P}_{Av} * \sqrt{\frac{m}{2 {kT}_{Av}}} * \frac{A h}{L} * ( \frac{Δ T}{{T}_{Av}} {M}_{t} - \frac{{Δ}_{p}}{{P}_{Av}} {M}_{p} ) \qquad ( 7 )$$

where m is the mass of one gas molecule to be driven by the pump; k is Boltzmann's constant for the gas; A is the cross-sectional area of a single channel, or multiple channels in parallel; h is channel height (for rectangular channel cross-sections) or diameter (for circular channel cross-sections); L is channel length; ΔT is temperature difference across the pump from hot side to cold side of the channel; T_{Av} is the average temperature; P_{Av} is the average pressure; Δp is the pressure difference across the channel; and Mₜ and Mₚ are flow coefficients of the particular gas, which are dependent on temperature and pressure, respectively. The computed values of these coefficients are found in F. Sharipov, “Non-isothermal gas flow through rectangular microchannels,” Journal of Micromechanics and Microengineering, vol. 9, pp. 394-401, 1999, which is hereby incorporated by reference in its entirety, particularly with reference to Sections 3-5.

Last, equation 8 is used to estimate ΔT across the pump:

$$Δ {T}_{pmp} = \frac{[ {R}_{pump} ] * {[ {R}_{middle} ]}^{*} [ Δ {T}_{total} ]}{[ {R}_{pump} + 2 {R}_{air} ] * [ {R}_{middle} + {R}_{bottom plate} + {R}_{heat sink} ]} \qquad ( 8 )$$

The estimated temperature difference is then used to calculate the pressure gradient using equation 1 or 1A (depending on the number of pumps). It is apparent that the heat sink has a significant impact on pump performance, showing that heat removal offers significant advantages in the pump's operation. This relationship is underscored by the fact that, as evident from equation 6, resistance of the heat sink is inversely proportional to ΔT.

Thermal circuit analysis is also useful for predicting dynamic changes in temperature difference and their effect on the pressure gradient as fluid level changes in the reservoir. Before pumping, the liquid in the reservoir will be at a full level, or some other predetermined level. If full, there will be no substantial space or gap for air between the pump and the liquid (for integrated devices), or otherwise within the reservoir. However, as liquid is driven out of the reservoir pneumatically, thermal resistance decreases.

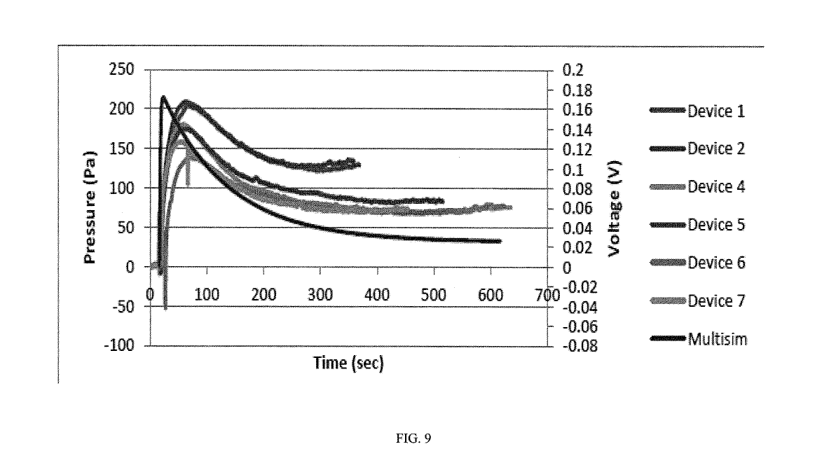

FIG. 8 depicts a thermal circuit associated with a fabricated cascaded device that was analyzed with the LabVIEW Multisim™ product. FIG. 8 provides a schematic and graphical capture and simulation environment for circuits and shows positions where thermal resistance affecting heat flow can be calculated. In some embodiments, these positions include the pump itself, the bottom plate, and the heat sink, for example, in addition to the gas-filled space around the pump. There are 17 resistors and 17 capacitors within the circuit shown in FIG. 8. In FIG. 8, for example, resistor R8 and capacitor C8, respectively, are the resistor and capacitor elements representing the nanoporous material of the pump, and ΔT was analyzed using equation 8 as shown to the right in the graph presented in FIG. 9 (noted there as voltage, which is analogous to ΔT).

Continuing with reference now to FIG. 9, pumps 1 to 7 were formed according to substantially the same parameters as pumps 1 and 2 as discussed above in connection with the testing results graphed in FIG. 7. Based on the current state of the art, slight manufacturing differences occurred in the fabrication of pumps 1 to 7 as referred to in FIG. 9, and these are associated with slight performance variance shown in the graph. What is validating here, though, is the experimental data of pumps 1-7 fairly overlays with the Multisim simulation. The data shows the relative temperature response of an embodiment as disclosed and claimed herein, when placed on the skin. Qualitatively, this data shows that temperature increases across the membrane sharply and peaks before settling over time to a substantially steady state temperature. Based on equation 1 and equation 1A, and as shown in FIG. 9, the steady state temperature corresponds to a steady state pressure, as well.

It will be understood that the embodiments described herein are not limited in their application to the details of the teachings and descriptions set forth, or as illustrated in the accompanying figures. Rather, it will be understood that the present embodiments and alternatives, as described and claimed herein, are capable of being practiced or carried out in various ways. Also, it is to be understood that words and phrases used herein are for the purpose of description and should not be regarded as limiting. The use herein of “including,” “comprising,” “e.g.,” “containing,” or “having” and variations of those words is meant to encompass the items listed thereafter, and equivalents of those, as well as additional items.

Accordingly, the foregoing descriptions of several embodiments and alternatives are meant to illustrate, rather than to serve as limits on the scope of what has been disclosed herein. The descriptions herein are not intended to be exhaustive, nor are they meant to limit the understanding of the embodiments to the precise forms disclosed. It will be understood by those having ordinary skill in the art that modifications and variations of these embodiments are reasonably possible in light of the above teachings and descriptions.

We claim:

1. A pneumatic pump configured to operate without electricity, comprising:
at least one channel with a first end and a second end, the second end being positioned closer to a heat source than the first end;
the at least one channel being configured to accept a gas from a pump inlet, the inlet being configured to allow the gas to enter the first end of the channel, and the channel being additionally configured to allow the gas to travel through the length of the channel and exit the channel at the second end;
wherein the first end corresponds to a cold side of the pump and the second end corresponds to a hot side of the pump, the hot and cold sides respectively being determined according to a temperature difference across the channel;
the channel having an average diameter of about 100 nm or less;
and wherein the at least one channel is configured to allow gas exiting at the second end to pass into a pump outlet.

2. The pump of claim 1, wherein the pump comprises a nanoporous material having a plurality of channels, each having a pore size of about 100 nm or less.

3. The pump of claim 2, wherein each of the plurality of channels has a pore size of about 25 nm.

4. The pump of claim 2, wherein the nanoporous material is selected from the group polypropylene, silica, nitrocellulose, aerogel, and mixed cellulose esters.

5. The pump of claim 1, wherein the pump is formed from a hydrophobic material.

6. The pump of claim 1, configured for operational attachment with a heat sink.

7. The pump of claim 1, wherein the heat source is produced by a human or animal.

8. The pump of claim 1, wherein the heat source is solar energy.

9. A pumping apparatus, comprising:
at least one pneumatic pump according to claim 1; and
a reservoir for storing a substance;
wherein the pump and the reservoir are in fluid communication.

10. The pumping apparatus of claim 9, wherein the pump comprises a nanoporous material having a plurality of channels.

11. The pumping apparatus of claim 9, wherein at least one pneumatic pump comprises a plurality of pumps connected in series.

12. The pumping apparatus of claim 9, further comprising outlet tubing in fluid communication with the reservoir and configured to permit the substance to travel therethrough during pneumatically-driven movement.

13. The pumping apparatus of claim 9, wherein the pump is positioned atop the reservoir.

14. The pumping apparatus of claim 9, further comprising a plate positioned between the hot side of the pump and a heat source.

15. The pumping apparatus of claim 14, further comprising a heat sink for increasing the temperature difference between the hot side and the cold side.

16. The pumping apparatus of claim 14, configured to deliver medication.

17. A device configured to pneumatically move a substance, comprising:
the pumping apparatus of claim 9; and
a plate positioned between the hot side of the pump and a heat source;
wherein the device is wearable by a human or animal.

18. The device of claim 17, further comprising a heat sink for increasing the temperature difference between the hot side and the cold side.

19. The device of claim 17, further comprising an elastic strap configured to be worn by a user.

20. The device of claim 17, configured to deliver medication.
